(19) United States
(12) Patent Application Publication
(10) Pub. No.: US 2021/0024901 A1
(43) Pub. Date: Jan. 28, 2021

(54) METHOD FOR CHANGING COENZYME ACTIVITY AND PREFERENCE OF GLUCOSE DEHYDROGENASE AND USE THEREOF

(71) Applicant: XIAMEN UNIVERSITY, Xiamen (CN)

(72) Inventors: Keju JING, Xiamen (CN); Yujiao SHEN, Xiamen (CN); Wei XIONG, Xiamen (CN); Xueping LING, Xiamen (CN); Yinghua LU, Xiamen (CN)

(21) Appl. No.: 16/825,652

(22) Filed: Mar. 20, 2020

(30) Foreign Application Priority Data
Jul. 24, 2019 (CN) 201910670475.4

(51) Int. Cl. C12N 9/04; C12N 15/10

(52) U.S. Cl. CPC C12N 9/0006; C12N 15/102; C12Y 101/9901

(57) ABSTRACT

The present invention provides a method for changing an coenzyme activity and a preference of glucose dehydrogenase and the use thereof, and relates to the technical field of genetic engineering. In the method of the present invention, by performing a site-directed mutation of the fourth amino acid in a conserved sequence GXXXGXG of the glucose dehydrogenase, a mutant protein with the changed coenzyme activity and preference can be simply and directly obtained without a large amount of screening, such that the enzyme can be widely applied to catalyze a coenzyme regeneration reaction. In the embodiments of the present invention, four glucose dehydrogenase mutants T17G, T17K, T17RGDH DN46 and K17G are finally obtained by site-directed mutation of the 4th amino acid (which binds with a nicotinamide coenzyme ribose 2′-phosphate) in the conserved sequence GXXXGXG widely existed in glucose dehydrogenases from different sources, and upon verification, the enzyme activity, preference and the like of them all have been varied.

### RELATED APPLICATION

The present application claims priority to Chinese Application No. 201910670475.4, filed Jul. 24, 2019, and is hereby incorporated by reference in its entirety into the present application.

### INCORPORATION BY REFERENCE

The sequence listing provided in the file entitled EFILED_SEQUENCE_LISTING_txt.txt, which is an ASCII text file that was created on Jul. 24, 2019, and which comprises 4096 bytes, is hereby incorporated by reference in its entirety.

### TECHNICAL FIELD

The present invention belongs to the technical field of genetic engineering, and particularly relates to a method for changing coenzyme activity and preference of glucose dehydrogenase and the use thereof.

### BACKGROUND

The redox enzymes are a family of enzymes that catalyze various chemical reactions with a highly efficient, stereoselective, and environment-friendly characteristic. Among all known redox enzymes, about 80% of them require nicotinamide adenine dinucleotide (NAD⁺/NADH) coenzyme for catalysis, 10% of them use nicotinamide adenine dinucleotide phosphate (NADP⁺/NADPH) as a coenzyme, and only a few of them use flavin (FMN/FAD) and ubiquinone CoQ (PQQ) as coenzymes. The nicotinamide coenzyme plays an electron transfer role in the redox reaction. As the reaction continues, the coenzyme will gradually be reduced to NAD(P)H, eventually leading to the suspension of the reaction. In order to enable the redox enzyme catalysis reaction to continue, supplementary NAD(P)⁺ must be continuously added. However, due to the high price of the coenzyme NAD(P)⁺, the industrialization of the redox enzyme catalysis will be limited if there is no effective method to regenerate the reductive coenzyme NAD(P)H.

At present, there are four methods for regenerating the coenzyme NAD(P)H: an enzymatic method, a chemical method, an electrochemical method, and a photochemical regeneration method. Among then, the enzymatic method has become a commonly-used coenzyme regeneration method due to its high regeneration efficiency and mild reaction conditions. The coenzyme regeneration via the enzymatic method requires the establishment of a second enzymatic reaction in which a second enzyme is used to reduced NAD(P)⁺ into NAD(P)H, forming a recycling system for coenzyme regeneration. The glucose dehydrogenase is widely used in a coenzyme regeneration system in a redox enzyme catalytic reaction due to its high activities on both of coenzymes NAD⁺/NADP⁺,

The glucose dehydrogenase (GDH) belongs to the short-chain dehydrogenases/reductases (SDR) family, and is a member of the ethanol dehydrogenase family. It can be widely used as an enzyme for the diagnosis of a blood glucose concentration and a biofuel cell. The GDH consists of four identical subunits, and has a size of about 28 KD. The glucose dehydrogenase (GDH), as a key enzyme in a pentose phosphate metabolism pathway, uses NAD(P)⁺ as a coenzyme and specifically catalyzes D-glucose to produce β-D-glucolactone. The GDH uses cheap glucose as the substrate, and the reaction product glucolactone will not pollute the environment. The GDH has great potential application value in the regeneration of the coenzyme NAD(P)H.

At present, the directed evolution research work on changing the coenzyme activity and the preference of the glucose dehydrogenase mainly focuses on the following two aspects: the first one is to establish a GDH gene mutation library by random mutation of a GDH gene, and then obtain a target mutant protein by high-throughput screening from the great number of mutant proteins; the second one is conducting saturation mutation from many amino acid residues in the coenzyme active site of the GDH, and then obtaining a target protein through a high-throughput screening method. Both of the above two methods need to create the massive mutant genes library through random mutagenesis of the GDH gene sequence. It will be a difficult task how to screen a target mutant protein from a large number of mutant libraries, and thus it is limited to apply these methods in actual production.

### SUMMARY

In view of this, an objective of the present invention is to provide a method for changing a coenzyme activity and a preference of glucose dehydrogenase and the use thereof, where by performing a site-directed mutation of the 4th amino acid in a conserved sequence (GXXXGXG) of the glucose dehydrogenase, a mutant protein with the changed coenzyme activity and preference can be efficiently and directly obtained without a large amount of screening, such that the enzyme can be widely applied to catalyze a coenzyme regeneration reaction.

In order to realize the aforementioned objective of the present invention, the present invention provides the following technical solution. The present invention provides a method for changing a coenzyme activity and preference of glucose dehydrogenase, including performing a site-directed mutation on the 4th amino acid in a conserved sequence of the glucose dehydrogenase.

Preferably, the conserved sequence of the glucose dehydrogenase is an active-site conserved sequence which binds with a nicotinamide coenzyme, and the amino acid sequence of the conserved sequence is as set out in SEQ ID NO. 1.

The present invention provides a glucose dehydrogenase mutant constructed by the method, where the amino acid sequence of the conserved sequence of the glucose dehydrogenase mutant is as set out in any one of SEQ ID Nos. 2-5.

The present invention also provides a mutation primer set for constructing the glucose dehydrogenase mutant, where the sequences of the mutation primer set are in SEQ ID Nos. 6-7, SEQ ID Nos. 8-9, SEQ ID Nos. 10-11, or SEQ ID Nos. 12-13.

The present invention also provides the use of the method or the glucose dehydrogenase mutant or the mutation primer set in a coenzyme regeneration system during a redox enzyme catalytic reaction.

The present invention also provides the use of the method or the glucose dehydrogenase mutant or the mutation primer set in a biofuel cell.

The present invention provides a method for changing a coenzyme activity and preference of glucose dehydrogenase, including performing a site-directed mutation on the 4th amino acid in a conserved sequence (GXXXGXG) of the glucose dehydrogenase. In the method of the present invention, by performing a site-directed mutation of the 4th amino acid in a conserved sequence of the glucose dehydrogenase, a mutant protein with the changed coenzyme activity and preference can be simply and directly obtained without a large amount of screening, such that the enzyme can be widely applied to catalyze a coenzyme regeneration reaction. In embodiments of the present invention, four glucose dehydrogenase mutants T17G, T17K, T17R and K17G are finally obtained by site-directed mutation of the 4th amino acid (which binds with the nicotinamide coenzyme ribose 2′-phosphate) in the conserved sequence (GXXXGXG) widely existed in the glucose dehydrogenase GDH DN46 (hereinafter referred to as GDH DN46 for short) derived from B. meaterium IWG3 and the glucose dehydrogenase GDH233 (hereinafter referred to as GDH233 for short) derived from B. meaterium AS1.223, and they are expressed in BL21 competent cells, purified and measured for enzyme activities. The results show that the mutant T17G has a decreased enzyme activity on NAD which has been decreased by about 24.8%, and an increased enzyme activity on NADP which has been increased by 28.1%, and meanwhile has a coenzyme preference which has been changed from a wild-type NAD to NADP⁺; as compared with the wild-type GDH DN46 , the mutant T17K has decreased enzyme activities on both of NAD⁺, NADP⁺, with the enzyme activities on NAD and NADP being decreased by 24.8% and 28.1%, respectively; as compared with the wild-type GDH DN46 , the mutant T17R has significantly decreased enzyme activities on NAD(P)+, with the enzyme activity on NAD being decreased by about 80%, and the enzyme activity on NADP being decreased by about 97%; and as compared with the wild-type GDH 223, the mutant K17G has varying degrees of improved enzyme activities on NAD(P)⁺, with the enzyme activity on NAD being increased by about 104.58%, and the enzyme activity on NADP being greatly increased, and the obtained single mutant K17G can be better applied in the regeneration of NAD(P)H.

### BRIEF DESCRIPTION OF THE DRAWINGS

FIGS. 1A and 1B show the expression and purification of a GDH DN46 mutant, where bands 1 and 2 are a crude enzyme and a pure enzyme of a wild-type GDH DN46, bands 3 and 4 are a crude enzyme and a pure enzyme of a mutant T17G, bands 5 and 6 are a crude enzyme and a pure enzyme of a mutant T17K, and bands 7 and 8 are a crude enzyme and a pure enzyme of a mutant T17R;

FIGS. 2A and 2B show the expression and purification of a GDH223 mutant K17G, where bands 1 and 2 are a crude enzyme and a pure enzyme of a wild-type GDH223, and bands 3 and 4 are a crude enzyme and a pure enzyme of a mutant K17G;

FIGS. 3A and 3B show the enzyme activity determination of the mutants;

FIG. 4 is a pattern diagram showing the principle of a coenzyme regeneration system; and

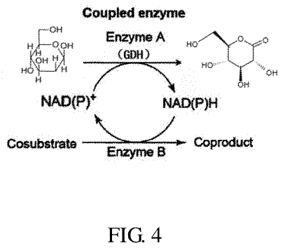

FIG. 5 is a pattern diagram showing the principle of a biofuel cell.

### DESCRIPTION OF THE EMBODIMENTS

The present invention provides a method for changing a coenzyme activity and a preference of glucose dehydrogenase, including performing a site-directed mutation on the 4th amino acid in a conserved sequence of the glucose dehydrogenase.

The conserved sequence of the glucose dehydrogenase is an active-site conserved sequence which binds with a nicotinamide coenzyme, the amino acid sequence of the conserved sequence is as set out in SEQ ID NO.1: GXXXGXG, the conserved sequence is 14→20 sites in the full amino acid sequence of the glucose dehydrogenase, and the site-directed mutation occurs at the 17th site.

The present invention provides a glucose dehydrogenase mutant constructed by the method, where the amino acid sequence of the conserved sequence of the glucose dehydrogenase mutant is as set out in any one of SEQ ID Nos. 2-5.

In the present invention, for convenience in explaining the solution of the present invention, in the embodiments of the present invention, taking the glucose dehydrogenase GDH DN46 (hereinafter referred to as GDH DN46 for short) derived from B. meaterium IWG3 and the glucose dehydrogenase GDH233 (hereinafter referred to as GDH223 for short) derived from B. meaterium AS1.223 as examples, the amino acid at the 17th site of each of GDH DN46 and GDH223 is subjected to site-directed mutation to construct three GDH DN46 mutants T17G, T17K and T17R and a GDH223 mutant K17G, but the specific claimed scope of the present invention should not be considered as limited to this. In the embodiments of the present invention, the amino acid sequence of the conserved sequence of the glucose dehydrogenase mutant is as shown in Table 1.

TABLE 1
Names and sequences of conserved sequences of
the mutants

| Mutant | SEQ ID NO | Sequence |
|---|---|---|
| T17G | 2 | GXXGGXG |
| T17K | 3 | GXXKGXG |
| T17R | 4 | GXXRGXG |
| K17G | 5 | GXXGGXG |

The present invention also provides a mutation primer set for constructing the glucose dehydrogenase mutant, where the sequences of the mutation primer set are as set out in SEQ ID Nos. 6-7, SEQ ID Nos. 8-9, SEQ ID Nos. 10-11, or SEQ ID Nos. 12-13.

In the present invention, the corresponding relationship between the mutation primer set and the glucose dehydrogenase mutants is as shown in Table 2.

TABLE 2
The corresponding relationship between the mutant
primers and the mutants

| | SEQ ID | |
|---|---|---|
| Mutant | NO | Sequence |
| T17G | 6 | F: CATAGACTTTCCCAGTCCACCAGAAGATC |
| | 7 | R: GGTGGACTGGGAAAGTCTATGGCTATTC |
| T17K | 8 | F: GACTTTCCCAGTCCCTTAGAAGATCCAG |
| | 9 | R: AGGGACTGGGAAAGTCTATGGCTATTCG |
| T17R | 10 | F: GACTTTCCCAGTCCTCTAGAAGATCCAG |
| | 11 | R: GAGGACTGGGAAAGTCTATGGCTATTCG |
| K17G | 12 | F: CATTGCGCGACCCAATCCTCCTGATCCGCC |
| | 13 | R: GGAGGATTGGGTCGCGCAATGGCCGTTC |

In the Table 2, the underlined part of the primer sequence is the mutation site.

In the present invention, when the mutation primer set is applied to carry out glucose dehydrogenase site-directed mutation, it is preferred that a recombinant plasmid is used as a template, and the recombinant plasmid is preferably obtained by using pET28a as a basic plasmid, and including the glucose dehydrogenase sequence onto the pET28a plasmid through an artificial synthesizing or cloning method. In the embodiments of the present invention, the two recombinant plasmids from different sources are named as pET28a-GDH DN46 and pET28a-GDH223, respectively.

In the present invention, the site-directed mutation is preferably carried out in a PCR manner, and the PCR system is preferably a 20pL system, which includes: 50-100 ng of a template DNA, each 1 μL of the mutation primers (10 μM), 10 μL of a Prime STAR Max DNA Polyase, and the balance of ddH₂O for making up 20 μL. The PCR procedure of the present invention preferably includes: pre-denaturing at 94° C. for 4 min; degenerating at 94° C. for 20 s, annealing at 62° C. for 20 s, extending at 72° C. for 3 min, 30 cycles; extending at 72° C. for 10 min; and storing at 4° C. In the present invention, after the PCR, it preferably further includes verifying whether the mutant plasmid has been successfully constructed. The present invention has no specific limitation on the verification method, and the PCR product is preferably subjected to analysis by agarose gel electrophoresis. In the present invention, the correct PCR product as verified is digested with a Dpnl enzyme at 37° C. for 2 h to degrade a non-mutant type plasmid template; the PCR product that has been digested with the Dpnl enzyme, is directly transformed into BL21 competent cells; and a transformed single colony is picked for PCR verification of the colony, and subjected to sequencing. In the present invention, after Escherichia coli BL21-pET28a-GDH DN46 and BL21-pET28a-GDH 223 are obtained by the aforementioned method, it preferably further includes the expression and purification of the mutants. The present invention has no specific limitation on the expression and purification methods, and preferably a IPTG induction method and a SDS-PAGE purification method are used.

The present invention also provides the use of the method or the glucose dehydrogenase mutant or the mutation primer set in a coenzyme regeneration system during a redox enzyme catalytic reaction. In the present invention, the regeneration, which mainly forms a coupling system with another enzyme that can utilize the coenzyme NADPH, can continuously produce NAD(P)⁺,

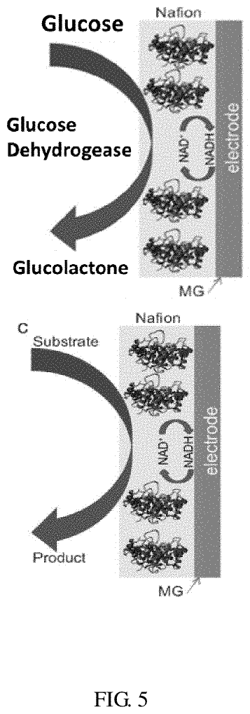

The present invention also provides the use of the method or the glucose dehydrogenase mutant or the mutation primer set in a biofuel cell. In the present invention, the principle of applying it into a biofuel cell is as shown in FIG. 5.

Hereafter the method for changing the coenzyme activity and the preference of glucose dehydrogenase and the use thereof as provided by the present invention will be described in detail in connection with the following examples, but they should not be construed as limiting the claimed scope of the present invention.

### EXAMPLE 1

Construction and Enzyme Activity Determination of the Mutant T17G

Escherichia coli BL21-pET28a-GDH DN46 was amplified by culturing in a LB medium at 37° C. for 16 h, so as to obtain BL21 bacterial cells. The bacterial cells were centrifuged at 13,000 rpm, and the bacterial cell precipitate is collected, and extracted for the plasmid pET28a- GDH DN46 , and the extracted plasmid was used as a template for PCR site-specific mutation to construct a mutant plasmid pET28a- GDH DN46 T17G. The mutation primers were as follows (5′-3′), where the underlined part was the mutation site:

IF
(SEQ ID NO. 6)
CATAGACTTTCCCAGTCCACCAGAAGATC
IR
(SEQ ID NO. 7)
GGTGGACTGGGAAAGTCTATGGCTATTC

The successfully constructed PCR product was enzymatically digested with the Dpnl enzyme at 37° C. for 2 hours, so as to digest a non-mutant plasmid template in the product. The digested product was directly transformed into BL21 competent cells. The transformed single colony was picked for PCR verification of the colony, and then subjected to sequencing. A correct sequencing result indicated that the mutant T17G had been successfully constructed.

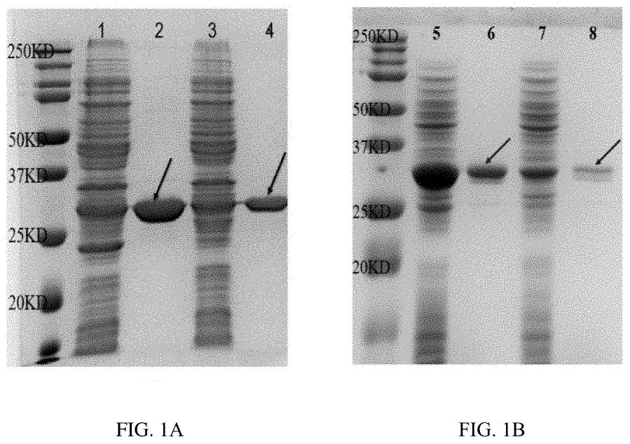

The recombinant Escherichia coli, which was successfully sequenced, was inoculated into a LB medium containing 50 g/mL kanamycin at the inoculation amount of 1%, subjected to shaking culture at 37° C. and 200 rpm for 3 h, then added with a IPTG inducer, incubated continually at the same temperature for 12 h, and then centrifuged at 7,000 rpm for 10 min to collect the precipitate as the obtained bacterial cells after induced expression. The bacterial cell precipitate was washed twice with PBS, and centrifuged under the same conditions as above. The resuspended bacterial solution was subjected to ultrasonic crushing under a condition of 200 W, where the ultrasonic crushing was operated for 3 sec, and then paused for 6 sec, and such a process was repeated for 90 times. After completion of the crushing, the crushed solution was centrifuged (10,000 rpm, 30 min) for separation, and the supernatant, a crude enzyme solution, was collected. The crude enzyme solution was purified by a nickel column, and the purification effect was as shown in bands 3 and 4 in the panel 1-a of FIG. 1. As compared with the crude enzyme solution, in the purified one most of the impure proteins had been removed, and the target protein accounted for more than 95% of the total protein, and thus the purified enzyme solution could be used for subsequent determination regarding the enzyme activity.

Enzyme Activity Determination of the Wild-Type Mutant.

The total reaction system was 220 μL, including 50 μL of 0.2M glucose, 11 μL of coenzymes (NAD⁺, or NADP⁺), 10 μL of a pure enzyme solution, and the balance of 20 mM PBS at the pH of 7.2 for making up 220 μL. The change of absorbance at 340 nm was determined in a microplate reader.

$$\begin{matrix}Enzyme Activity ( \frac{U}{mL} ) = \frac{Δ A \text{/} Δ t \cdot Vs}{V t \cdot L \cdot ɛ} & ( 1 ) \\ Specific Enzyme Activity ( \frac{U}{mg} ) = \frac{Enzyme Activity}{Protein Concentration} & ( 2 )\end{matrix}$$

where Vs represents the total reaction volume (mL); Vt represents the enzyme volume (ml); ΔA represents the absorbance change value; At represents the time of absorbance changing (min); L represents an optical path (cm); ε represents a molar absorption coefficient (6.22 ml (μ mol cm)⁻¹).

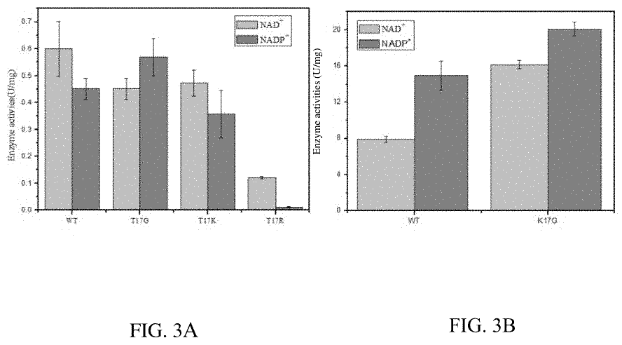

The results were as shown in the panel 3-a of FIG. 3, and as compared with the wild-type GDH DN46 , the mutant T17G had a decreased enzyme activity on NAD⁺ which had been decreased by about 24.8%, and had an increased enzyme activity on NADP⁺ which had been increased by 28.1%. At the same time, the coenzyme preference was changed from the wild-type NAD⁺ to NADP⁺,

### EXAMPLE 2

Construction and Enzyme Activity Determination of the Mutant T17K

Except that the mutation primer was different from that of Example 1, the other conditions are the same as those of Example 1: the mutation primers for constructing the mutant plasmid pET28a- GDH DN46 T17K was as set out below (5′-3′), where the underlined part was the mutation site:

IF
(SEQ ID NO. 8)
GACTTTCCCAGTCCCTTAGAAGATCCAG
IR
(SEQ ID NO. 9)
AGGGACTGGGAAAGTCTATGGCTATTCG

The collected supernatant crude enzyme solution was purified, and the purification effect was as shown in bands 5 and 6 in the panel 1-b of FIG. 1. As compared with the crude enzyme solution, in the purified one most of the impure proteins had been removed, and the target protein accounted for more than 95% of the total protein, and thus the purified enzyme solution could be used for subsequent determination regarding the enzyme activity.

The determined enzyme activity data was as shown in the panel 3-a of FIG. 3, and as compared with the wild-type GDH DN46 , the mutant T17K had decreased enzyme activities on both NAD⁺ and NADP⁺, with the enzyme activities on NAD⁺ and NADP⁺ being decreased by 24.8% and 28.1%, respectively.

### EXAMPLE 3

Construction and Enzyme Activity Determination of the Mutant T17R

Except that the mutation primer was different from that of Example 1, the other conditions are the same as those of Example 1: the mutation primers for constructing the mutant plasmid pET28a- GDH DN46 T17R was as set out below (5′-3′), where the underlined part was the mutation site:

IF
(SEQ ID NO. 10)
GACTTTCCCAGTCCTCTAGAAGATCCAG
IR
(SEQ ID NO. 11)
GAGGACTGGGAAAGTCTATGGCTATTCG

The collected supernatant crude enzyme solution was purified, and the purification effect was as shown in bands 7 and 8 in the panel 1-b of FIG. 1. As compared with the crude enzyme solution, in the purified one most of the impure proteins had been removed, and the target protein accounted for more than 95% of the total protein, and thus the purified enzyme solution could be used for subsequent determination regarding the enzyme activity.

The determined enzyme activity data was as shown in the panel 3-a of FIG. 3, and as compared with the wild-type GDH DN46, the mutant T17R had significantly decreased enzyme activities on NAD(P)⁺, with its enzyme activity on NAD being decreased by about 80%, and its enzyme activity on NADP being decreased by about 97%.

### EXAMPLE 4

Construction and Enzyme Activity Determination of the Mutant K17G

The original bacterial strain used in this example was different from that of the example 1. In this example, Escherichia coli BL21-pET28a-GDH 223 was used, and a mutant plasmid pET28a- GDH DN46 T17G was constructed by PCR site-directed mutation. The mutation primers were as set out below (5′-3′), where the underlined part was the mutation site:

IF
(SEQ ID NO. 12)
CATTGCGCGACCCAATCCTCCTGATCCGCC
IR
(SEQ ID NO. 13)
GGAGGATTGGGTCGCGCAATGGCCGTTC

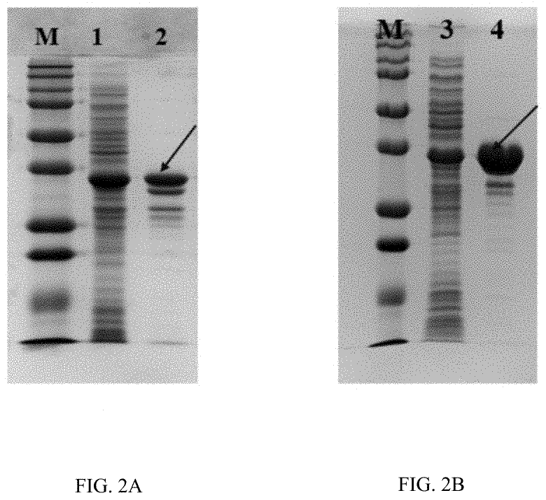

The collected supernatant crude enzyme solution was purified, and the purification effect was as shown in bands 3 and 4 in the panel 2-b FIG. 2. As compared with the crude enzyme solution, in the purified one most of the impure proteins had been removed, and the target protein accounted for more than 95% of the total protein, and thus the purified enzyme solution could be used for subsequent determination regarding the enzyme activity.

The determined enzyme activity data was as shown in the panel 3-a of FIG. 3, and as compared with the wild-type GDH 223, the mutant K17G had varying degrees of improved enzyme activities on NAD(P)⁺, with its enzyme activity on NAD being increased by about 104.58%, and its enzyme activity on NADP being significantly increased. The obtained single mutant K17G could be better applied in the regeneration of NAD(P)H.

The present invention provides a method for changing a coenzyme activity and a preference of glucose dehydrogenase and the use thereof, where by performing a site-directed mutation of the fourth amino acid in the conserved sequence GXXXGXG of the glucose dehydrogenase, a mutant protein with the changed coenzyme activity and preference can be simply and directly obtained without a large amount of screening, such that the enzyme can be widely applied to catalyze a coenzyme regeneration reaction.

The above description is only preferred embodiments of the present invention. It should be pointed out that, for those of ordinary skills in the art, several improvements and modifications can be made without departing from the principle of the present invention. These improvements and modifications should also be considered as falling into the claimed scope of the present invention.

1. A method for changing a coenzyme activity and a preference of glucose dehydrogenase, comprising performing a site-directed mutation on the 4th amino acid in a conserved sequence of the glucose dehydrogenase.

2. The method according to claim 1, wherein the conserved sequence of the glucose dehydrogenase is an active-site conserved sequence which binds with a nicotinamide coenzyme, and the amino acid sequence of the conserved sequence is as set out in SEQ ID NO. 1.

3. A glucose dehydrogenase mutant constructed by the method according to claim 1, wherein the amino acid sequence of the conserved sequence of the glucose dehydrogenase mutant is as set out in any one of SEQ ID Nos. 2-5.

4. A mutation primer set for constructing the glucose dehydrogenase mutant according to claim 3, wherein the sequences of the mutation primer set are as set out in SEQ ID Nos. 6-7, SEQ ID Nos. 8-9, SEQ ID Nos. 10-11, or SEQ ID Nos. 12-13.

5. The use of the method according to claim 1 in a coenzyme regeneration system during a redox enzyme catalytic reaction.

6. The use of the method according to claim 1 in a biofuel cell.

7. A glucose dehydrogenase mutant constructed by the method according to claim 2, wherein the amino acid sequence of the conserved sequence of the glucose dehydrogenase mutant is as set out in any one of SEQ ID Nos. 2-5.

8. A mutation primer set for constructing the glucose dehydrogenase mutant according to claim 4, wherein the sequences of the mutation primer set are as set out in SEQ ID Nos. 6-7, SEQ ID Nos. 8-9, SEQ ID Nos. 10-11, or SEQ ID Nos. 12-13.

9. The use of the method according to claim 2 in a coenzyme regeneration system during a redox enzyme catalytic reaction.

10. The use of the glucose dehydrogenase mutant according to claim 3 in a coenzyme regeneration system during a redox enzyme catalytic reaction.

11. The use of the glucose dehydrogenase mutant according to claim 4 in a coenzyme regeneration system during a redox enzyme catalytic reaction.

12. The use of the mutation primer set according to claim 5 in a coenzyme regeneration system during a redox enzyme catalytic reaction.

13. The use of the mutation primer set according to claim 6 in a coenzyme regeneration system during a redox enzyme catalytic reaction.

14. The use of the method according to claim 2 in a biofuel cell.

15. The use of the glucose dehydrogenase mutant according to claim 3 in a biofuel cell.

16. The use of the glucose dehydrogenase mutant according to claim 4 in a biofuel cell.

17. The use of the mutation primer set according to claim 5 in a biofuel cell.

18. The use of the mutation primer set according to claim 6 in a biofuel cell.


SEQUENCE LISTING (13 sequences)

SEQ ID NO: 1 - protein, 7 aa, artificial sequence
  the amino acid sequence of the conserved sequence
  misc_feature (2)..(4): Xaa can be any naturally occurring amino acid
  misc_feature (6)..(6): Xaa can be any naturally occurring amino acid
GXXXGXG

SEQ ID NO: 2 - protein, 7 aa, artificial sequence
  T17G
  misc_feature (2)..(3): Xaa can be any naturally occurring amino acid
  misc_feature (6)..(6): Xaa can be any naturally occurring amino acid
GXXGGXG

SEQ ID NO: 3 - protein, 7 aa, artificial sequence
  T17K
  misc_feature (2)..(3): Xaa can be any naturally occurring amino acid
  misc_feature (6)..(6): Xaa can be any naturally occurring amino acid
GXXKGXG

SEQ ID NO: 4 - protein, 7 aa, artificial sequence
  T17R
  misc_feature (2)..(3): Xaa can be any naturally occurring amino acid
  misc_feature (6)..(6): Xaa can be any naturally occurring amino acid
GXXRGXG

SEQ ID NO: 5 - protein, 7 aa, artificial sequence
  K17G
  misc_feature (2)..(3): Xaa can be any naturally occurring amino acid
  misc_feature (6)..(6): Xaa can be any naturally occurring amino acid
GXXGGXG

SEQ ID NO: 6 - DNA, 29 bp, artificial sequence
  T17G F
catagactttcccagtccaccagaagatc

SEQ ID NO: 7 - DNA, 28 bp, artificial sequence
  T17G R
ggtggactgggaaagtctatggctattc

SEQ ID NO: 8 - protein, 28 aa, artificial sequence
  T17K F
GACTTTCCCAGTCCCTTAGAAGATCCAG

SEQ ID NO: 9 - DNA, 28 bp, artificial sequence
  T17K R
agggactgggaaagtctatggctattcg

SEQ ID NO: 10 - DNA, 28 bp, artificial sequence
  T17R F
gactttcccagtcctctagaagatccag

SEQ ID NO: 11 - DNA, 28 bp, artificial sequence
  T17R R
gaggactgggaaagtctatggctattcg

SEQ ID NO: 12 - DNA, 30 bp, artificial sequence
  K17G F
cattgcgcgacccaatcctcctgatccgcc

SEQ ID NO: 13 - DNA, 28 bp, artificial sequence
  K17G R
ggaggattgggtcgcgcaatggccgttc